(19) United States
(12) Patent Application Publication
(10) Pub. No.: US 2026/0182911 A1
(43) Pub. Date: Jul. 2, 2026

(54) EXOSKELETON, METHOD OF CONTROLLING THEREOF, AND TREMOR SIMULATOR DEVICE

(71) Applicant: NANYANG TECHNOLOGICAL UNIVERSITY, Singapore (SG)

(72) Inventors: Yifan WANG, Singapore (SG); Guohui WANG, Singapore (SG)

(21) Appl. No.: 18/868,032

(22) PCT Filed: Jun. 14, 2023
(86) PCT No.: PCT/SG2023/050422

§ 371 Date: Nov. 21, 2024

(30) Foreign Application Priority Data
Jun. 14, 2022 (SG) 10202250155H

(51) Int. Cl. A61B 5/00; A61B 5/11; B33Y 80/00

(52) U.S. Cl. CPC A61B 5/4836; A61B 5/1101; A61B 5/4082; A61B 5/6812; B33Y 80/00; A61B 2560/0462

(57) ABSTRACT

The present application provides embodiments of exoskeletons, methods of controlling thereof, and tremor simulator devices. According to an embodiment, there is provided an exoskeleton. The exoskeleton comprises a first support; a second support; a joint structure rotatably connecting the first support and the second support; and a controller. The second support comprises: a first anchor component connecting to the joint structure, a second anchor component, and a sensor coupled to the controller, wherein the first anchor component and the second anchor component are connected by a variable stiffness unit coupled to the controller, and wherein the variable stiffness unit is configured to provide a variable damping force determined by the controller to reduce an amplitude of a tremor sensed by the sensor.

### TECHNICAL FIELD

The present specification relates broadly, but not exclusively, to exoskeletons, methods of controlling thereof, and tremor simulator devices.

### BACKGROUND

Tremor is a neurological disorder that causes shaking movements in one or more parts of a body, most often in hands. It can also occur in arms, legs, head, vocal cords, torso, etc. Its rhythmic pattern is caused by unintentional (involuntary) muscle contractions.

Tremor can occur sporadically on its own, or happen as a result of another disorder. One example of such disorder is Parkinson's disease (PD). Tremors affect approximately 80% of PD patients. It appears as unconscious shaking of the hand or arm muscles, which is exacerbated at rest or during times of emotional stress. When tremor occurs frequently, PD patients may lose perceptual function of the hand which may lead to paralysis. Furthermore, tremors can result in severe psychological burdens for the patients, such as depression, anxiety, impulsiveness, and dizziness. As a result, tremor suppression is critical for improving PD patients' daily lives and lowering their psychological burden.

As a non-invasive solution, exoskeletons that can suppress the tremors have attracted a lot of interest. Based on their working mechanisms, these exoskeletons can be classified into three major categories: active systems, passive systems, and semi-active systems.

Active systems actively provide forces or torques in the opposite direction of the tremor movement to reduce tremor vibration amplitude. Typical examples include wearable orthosis for tremor assessment and suppression (WOTAS) developed by Eduardo Rocon et al., tremor suppression orthosis (TSO) developed by Gil Herrnstadt et. al., permanent magnet linear motors (PMLMs) developed by Amir Hosein-Zamanian et. al., and wearable tremor suppression glove developed by Yue Zhou et al., These active systems apply effective dynamic forces to the upper limb shaking area and change its biomechanical properties in real-time with active control. However, most of these active systems use rigid exoskeletons driven by electric motors or other actuators. As a result, their weights are usually measured in kilograms which adds a heavy burden to the patient. Furthermore, rigid exoskeletons have poor compliance to the human wrist and provide low comfort levels when used in practice.

Passive systems typically suppress tremors by absorbing or dissipating the vibration energy. In 1998, Kotovsky et. al created Viscous Beam, the first passive device for vibration suppression, which limits wrist rotation by applying viscous damping to the flexion & extension of the wrist joint. Nicolas Philip Fromme et. al created a lightweight passive orthosis that uses an airbag to reduce wrist vibration. These passive systems can be very lightweight (minimum weight only at 33 g) compared to active and semi-active systems. However, the damping forces or torques provided by passive systems are typically very small, making it difficult to suppress the tremor effectively. Although Sreekanth Rudraraju et. al proposed a mass-spring-damper system that reduced tremor by 70% to 80%, such a system can only suppress the tremor in one direction.

Semi-active systems typically measure vibration amplitudes using embedded sensors and then adjust the damping force to suppress tremors. To generate damping force, the semi-active systems generally employ magnetorheological fluid, electromagnetic brake, pneumatic cylinder, etc. Among them, magnetorheological fluid is the most effective solution. The viscosity of a magnetorheological fluid can be continuously controlled with an applied magnetic field. It is typically lighter than the active system (approximately 200 g to 700 g) and eliminates the need for driving components such as motors, hence is better suited to the human wrist. However, the existing magnetic fluid-based flexible wrist exoskeleton mechanism can only provide about 10 N damping force in general, making it difficult to meet the practical needs.

A need therefore exists to provide a semi-active exoskeleton system that seeks to overcome or at least minimize the above-mentioned problems. In this regard, the present application provides exoskeletons that are not only lightweight but also able to produce a sufficient damping force that can effectively suppress tremors in multiple directions simultaneously, and methods of controlling thereof. Also provided is a tremor simulator device that can be used to simulate tremors in various directions that facilitate to, e.g. investigate effectiveness of the exoskeletons as disclosed herein.

### SUMMARY

According to an embodiment, there is provided an exoskeleton. The exoskeleton comprises a first support; a second support; a joint structure rotatably connecting the first support and the second support; and a controller. The second support comprises: a first anchor component connecting to the joint structure, a second anchor component, and a sensor coupled to the controller, wherein the first anchor component and the second anchor component are connected by a variable stiffness unit coupled to the controller, and wherein the variable stiffness unit is configured to provide a variable damping force determined by the controller to reduce an amplitude of a tremor sensed by the sensor.

According to another embodiment, there is provided a method of controlling an exoskeleton, the exoskeleton comprising a first support, a second support, a joint structure rotatably connecting the first support and the second support, and a controller, wherein the second support comprises a first anchor component connecting to the joint structure, a second anchor component, and a sensor coupled to the controller, and wherein the first anchor component and the second anchor component are connected by a variable stiffness unit coupled to the controller. The method comprises: sensing a tremor by the sensor, determining a variable damping force by the controller in response to the sensed tremor, and providing the variable damping force by the variable stiffness unit to reduce an amplitude of the tremor.

According to yet another embodiment, there is provided a tremor simulator device. The tremor simulator device comprises a linkage mechanism; a drive motor; and a speed controller. The linkage mechanism comprises a first rod and second rod that are configured to rotate with respect to each other, and a third rod fixedly connected to the second rod, wherein the third rod is configured to make a linear reciprocal motion with respect to the second rod, and wherein the speed controller is configured to control the drive motor to drive the first rod and the second rod to rotate and the third rod to make the linear reciprocal motion so as to cause a shaking at an end of a limb coupled to the tremor simulator device.

### BRIEF DESCRIPTION OF THE DRAWINGS

Embodiments and implementations are provided by way of example only, and will be better understood and readily apparent to one of ordinary skill in the art from the following written description, read in conjunction with the drawings, in which:

FIG. 1 shows a schematic diagram of an exoskeleton 100, according to an embodiment.

FIG. 2 shows a diagram 200 depicting another embodiment of an exoskeleton. In the embodiment, the exoskeleton is coupled to a wearer's arm and wrist. It is understandable to those skilled in the art that the exoskeleton can be coupled to other parts of the body of the wearer based on the practical needs.

FIG. 3 shows a diagram 300 depicting another embodiment of an exoskeleton. In this embodiment, the exoskeleton further comprises a vacuum pump provided at the waist of the wearer and connected to other components of the exoskeleton provided at the arm and wrist. It is understandable to those skilled in the art that the vacuum pump can be provided at other parts of the body of the wearer based on the practical needs.

FIG. 4a shows a diagram 400 of human wrist movements.

FIG. 4b shows a diagram 450 illustrating major muscles and bones involved in wrist tremor.

FIG. 5 shows a diagram 500 depicting an embodiment of a chain mail-like structured fabric, a diagram 510 depicting a morphology of the chain mail-like structured fabric in an unjammed soft state, and a diagram 520 depicting a morphology of the chain mail-like structured fabric in a jammed stiff state.

FIG. 6 is a flow chart illustrating a method 600 of controlling an exoskeleton, according to an embodiment.

FIG. 7 shows a schematic diagram of a control framework 700 to implement the method 600 of controlling an exoskeleton, according to an embodiment.

FIG. 8a shows a schematic diagram of a Three-Point bending test apparatus 800 that can be used to test damping forces provided by various embodiments of the exoskeleton of the present application in different directions.

FIGS. 8b, 8c and 8d respectively depict deformations of the variable stiffness unit of various embodiments of the exoskeleton during wrist extension, flexion, and abduction/adduction.

FIG. 9a shows a schematic diagram of a torsional testing system 900 that can be used to test damping torque provided by various embodiments of the exoskeleton of the present application.

FIG. 9b shows an embodiment of a torsional testing platform 902 used in the torsional testing system 900.

FIG. 10a shows a graph diagram depicting experimental results of damping force against rotation angle in extension direction under different working negative pressures.

FIG. 10b shows a graph diagram depicting experimental results of damping force against rotation angle in flexion direction under different working negative pressures.

FIG. 10c shows a graph diagram depicting experimental results of damping force against rotation angle in abduction/adduction direction under different working negative pressures.

FIG. 10d shows a graph diagram depicting experimental results of damping force against rotation angle in pronation/supination direction under different working negative pressures.

FIG. 11a shows a bar graph depicting critical tremor angles at various negative operating pressures in extension direction.

FIG. 11b shows a bar graph depicting critical tremor angles at various negative operating pressures in flexion direction.

FIG. 11c shows a bar graph depicting critical tremor angles at various negative operating pressures in abduction direction.

FIG. 11d shows a bar graph depicting critical tremor angles at various negative operating pressures in adduction direction.

FIG. 11e shows a bar graph depicting critical tremor angles at various negative operating pressures in pronation direction.

FIG. 11f shows a bar graph depicting critical tremor angles at various negative operating pressures in supination direction.

FIG. 12 shows a diagram 1200 depicting another embodiment of an exoskeleton in an experimental scene.

FIG. 13a shows a graph diagram depicting amplitude-frequency characteristics of wrist tremors in extension and flexion directions at various operating negative pressures.

FIG. 13b shows a graph diagram depicting amplitude-frequency characteristics of wrist tremors in abduction and adduction directions at various operating negative pressures.

FIG. 13c shows a graph diagram depicting amplitude-frequency characteristics of wrist tremors in pronation and supination directions at various operating negative pressures.

FIG. 13d shows a graph diagram depicting electromyography (EMG) signals during extension and flexion tremor at various operating negative pressures.

FIG. 13e shows a graph diagram depicting EMG signals during abduction and adduction tremor at various operating negative pressures.

FIG. 13f shows a graph diagram depicting EMG signals during pronation and supination tremor at various operating negative pressures.

FIG. 14a shows a schematic diagram of a tremor simulator device, according to an embodiment.

FIG. 14b shows an embodiment of a wire sensor and its testing principle.

FIG. 14c shows an embodiment of the tremor simulator device.

FIG. 15a shows a graph diagram depicting maximum displacement variations of the wrist under different operating negative pressures for the wrist in the extension and flexion direction at a tremor frequency in the range of 2.2 Hz to 2.7 Hz.

FIG. 15b shows a graph diagram depicting maximum displacement variations of the wrist under different operating negative pressures for the wrist in the extension and flexion direction at a tremor frequency in the range of 3.4 Hz to 3.7 Hz.

Skilled artisans will appreciate that elements in the figures are illustrated for simplicity and clarity and have not necessarily been depicted to scale. For example, the dimensions of some of the elements in the illustrations, block diagrams or flowcharts may be exaggerated in respect to other elements to help to improve understanding of the present embodiments.

### DETAILED DESCRIPTION

Embodiments will be described, by way of example only, with reference to the drawings. Like reference numerals and characters in the drawings refer to like elements or equivalents.

Embodiments of the present application provide exoskeletons that are not only lightweight but also able to produce a sufficient damping force that can effectively suppress tremors in multiple directions simultaneously, and methods of controlling thereof. Also provided is a tremor simulator device that can be used to simulate tremors in various directions that facilitate to, e.g., investigate effectiveness of the exoskeletons as disclosed herein.

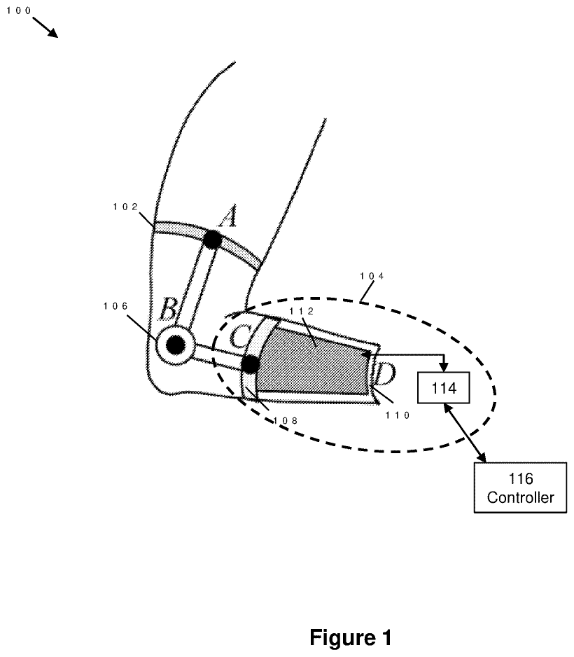

FIG. 1 illustrates a schematic diagram of an exoskeleton 100, according to an embodiment. In this embodiment, the exoskeleton 100 comprises a first support 102, a second support 104, a controller 116, and a joint structure 106 rotatably connecting the first support 102 and the second support 104. The second support 104 comprises a first anchor component 108, a second anchor component 110, and a sensor 114 coupled to the controller 116. In the second support 104, it is the first anchor component 108 that is connected to the joint structure 106.

In the embodiment shown in FIG. 1, the joint structure 106 comprises two arm members. Each of the two arm members has a first end securely engaging the first support 102 or the second support 104, i.e. at the first anchor component 108, respectively. Each of the two arm members has a second end rotatably interconnected to each other, which in turn provides a rotatable connection between the first support 102 and the second support 104.

In some embodiments, the rotatable interconnection of the two arm members is realised by a separate rotatable component. In other embodiments, the rotatable interconnection of the two arm members can be realised without a separate rotatable component. For example, the rotatable interconnection of the two arm members can be realised by the second ends of the two arm members coupling with each other to form an integral connection that allows certain degree of angular movement around the coupling point. It is understandable to those skilled in the art that the rotatable interconnection of the two arm members and in turn, the rotatable connection between the first support 102 and the second support 104 are not limited to the above described embodiments and can be realised in other manners based on the practical requirements.

In alternative embodiments, instead of two arm members, the joint structure 106 can comprise one single arm member having a first end securely engaging the first support 102 and a second end securely engaging the second support 104, i.e. at the first anchor component 108. The arm member needs to possess certain robustness to sustain tremors as well as certain flexibility to allow it to bend as a user bends its limb where the exoskeleton is located.

It is understandable to those skilled in the art that the shape and configuration of the joint structure 106 are not limited to the above described embodiments and can be realised in other manners based on the practical requirements.

In the embodiments, the secure engagement between the arm member(s) of the joint structure 106 with the first support 102 and the second support 104 (in particular, the first anchor component 108) can be a fixed attachment or a removable attachment. The fixed attachment can be realised by a bolt and nut mechanism, by welding, by a strong adhesive, or by other mechanisms that are appreciable to those skilled in the art.

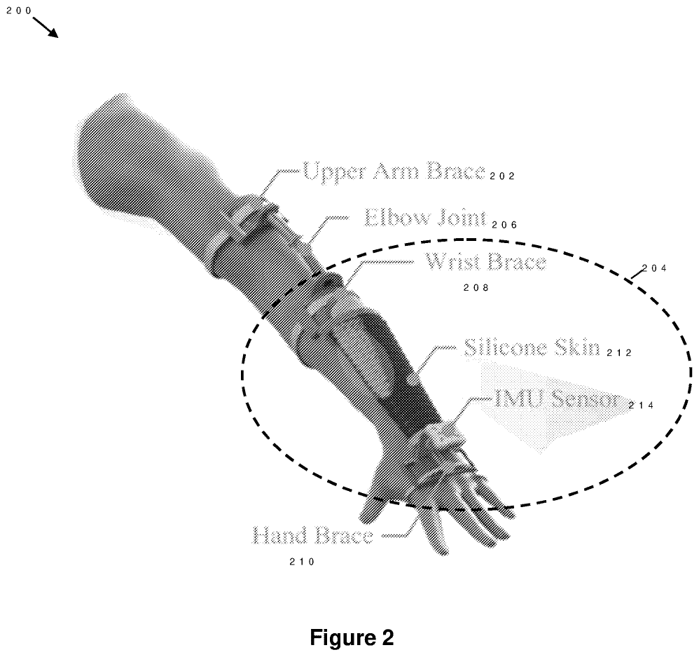

As for the removable attachment, an embodiment is depicted in FIG. 2. In this embodiment, the joint structure 106 comprises two arm members. Each of the two arm members comprises a brace structure on the first end. The first support 102 and the first anchor component 108 of the second support 104 can be disposed through or above the brace structures and fasten the brace structures so as to form secure engagements. Such secure engagements can be removed by loosening the first support 102 and the second support 104 (in particular, the first anchor component 108). In this embodiment and other embodiments of the present specification, the first support 102 and the second support 104 can be made of various materials, e.g., elastic straps, or lightweight metals and alloy alternatives such as aluminium, titanium, and magnesium, etc. Such a removable attachment facilitates to provide better portability and easy assemblability of the exoskeleton and allows the location or position of the exoskeleton to be adjusted by adjusting the brace structures thus providing more convenience to the user.

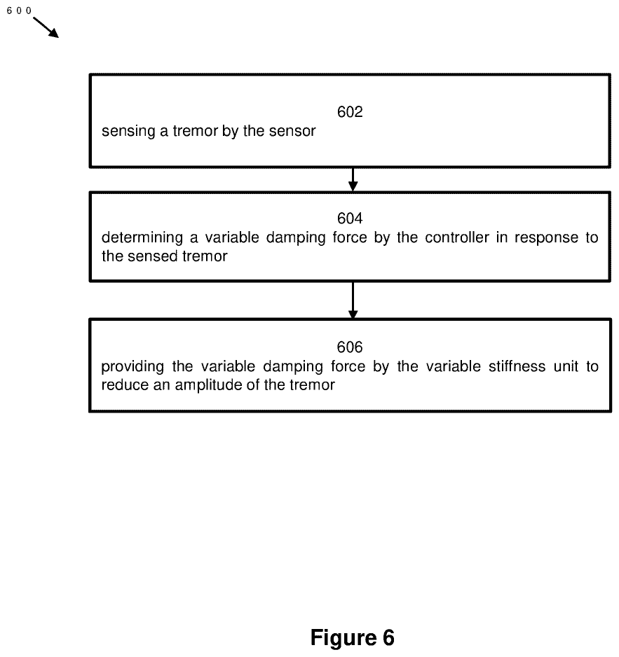

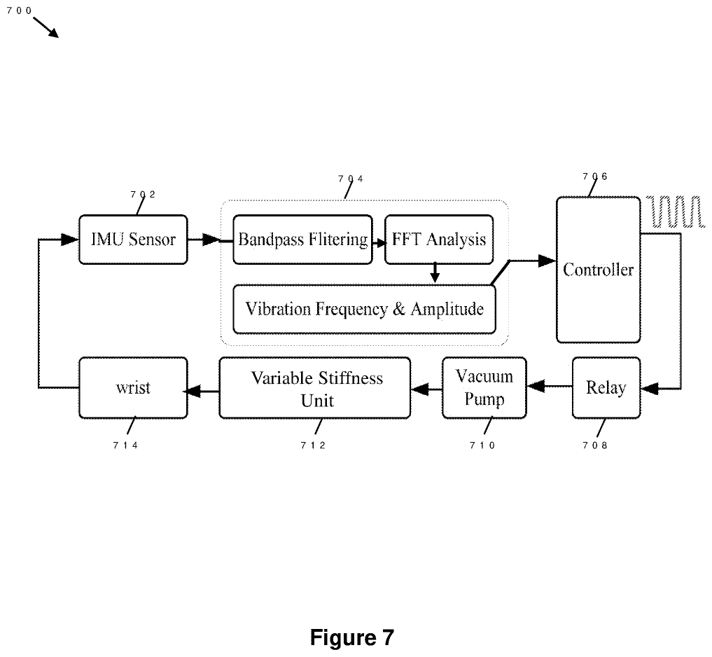

In the second support 104, the first anchor component 108 and the second anchor component 110 are connected by a variable stiffness unit 112 coupled to the controller 116. The variable stiffness unit 112 is configured to provide a variable damping force determined by the controller 116 to reduce an amplitude of a tremor sensed by the sensor 114. The provision of the variable damping force will be described in the following paragraphs, especially with respect to FIGS. 6 and 7.

Referring to FIG. 6, an exemplary method 600 of controlling the exoskeleton 100 so as to provide the variable damping force is depicted. The method 600 comprises at least the following steps.

In step 602, a tremor is sensed by the sensor 114.

In response to the sensed tremor, in step 604, the controller 116 is configured to determine a variable damping force.

Thereafter, in step 606, the variable damping force is provided by the variable stiffness unit 112 to reduce the amplitude of the tremor.

In some embodiments, the exoskeleton 100 can further comprise a vacuum pump. In these embodiments, the method 600 can further include the following steps.
detecting, by the controller 116, a tremor signal received from the sensor 114 upon sensing of the tremor at step 602; and
controlling, by the controller 116, the vacuum pump to activate the variable stiffness unit 112 to provide the variable damping force.

That is, in response to detection of a tremor signal received by the controller 116 from the sensor 114 upon sensing the tremor, the controller 116 is configured to control the vacuum pump to activate the variable stiffness unit 112 to provide the variable damping force.

It is appreciable to those skilled in the art that, aside from the vacuum pump, the variable stiffness unit 112 may be activated by other mechanisms to provide the variable damping force. For example, alternative ways of activating the variable stiffness unit include using strong electric or magnetic fields, changing temperatures, etc.

In FIG. 2, an embodiment 200 is depicted, in which the exoskeleton 100 is coupled to a user's arm and wrist. In this specification, a user is interchangeably referred to as a wearer. As described above, it is understandable to those skilled in the art that the exoskeleton 100 can be coupled to other parts of the body of the wearer based on the practical needs.

In the embodiment 200, the first support is couplable to an upper arm of a wearer of the exoskeleton 100. The first anchor component of the second support 204 is couplable to a forearm of the wearer of the exoskeleton 100. In this scenario, the joint structure 106 as shown in FIG. 1 is an elbow joint 206 that comprises two arm members. Each of the two arm members comprises a brace structure, i.e. upper arm brace 202 or wrist brace 208, respectively, on the first end. The first support and the first anchor component of the second support 104 can be disposed through or above the brace structures 202, 208 and fasten the brace structures 202, 208 so as to form secure engagements. Such secure engagements can be removed by loosening the first support 102 and the second support 104 (in particular, the first anchor component 108). In this embodiment and other embodiments of the present specification, the first support 102 and the second support 104 can be made of various materials, e.g., elastic straps, or lightweight metals and alloy alternatives such as aluminium, titanium, and magnesium.

In the embodiment 200, the second anchor component 214 of the second support 204 is couplable to a hand of the wearer of the exoskeleton, e.g. through a hand brace 210. The sensor 214 is configured to be located on or proximate to the hand. For example, the sensor 214 can be an inertial measurement unit (IMU) sensor.

In the embodiment 200, the variable stiffness unit 212 is located at or proximate to a wrist of the wearer of the exoskeleton, spanning from the first anchor component 208 to the second anchor component 214. Such an arrangement facilitates to provide a large coverage for the variable stiffness unit 212, thereby providing a sufficient damping force to reduce the tremors sensed in the user's arm and wrist. It is appreciable to those skilled in the art that the coverage of the variable stiffness unit 212 may be modified to a larger or smaller coverage based on the practical tremor suppression requirement. For example, if the tremors are not severe in a particular user, the configuration of the variable stiffness unit 212 may be modified by taking up just a portion of the area spanning from the first anchor component 208 to the second anchor component 214. The variable stiffness unit 212 can include an air-tight silicone skin wrapping two layers of chain mail-like structured fabrics (details of which will be described below). It is important to note that even when the the exoskeleton 100 is working, the user may prefer a slight wrist flexion to allow for movements such as gripping. As a result, the damping force provided by the the exoskeleton 100 in flexion must be slightly lower than in other directions. Based on this, an arch structure is created that fits to the back of the arm which enables bending in the flexion direction.

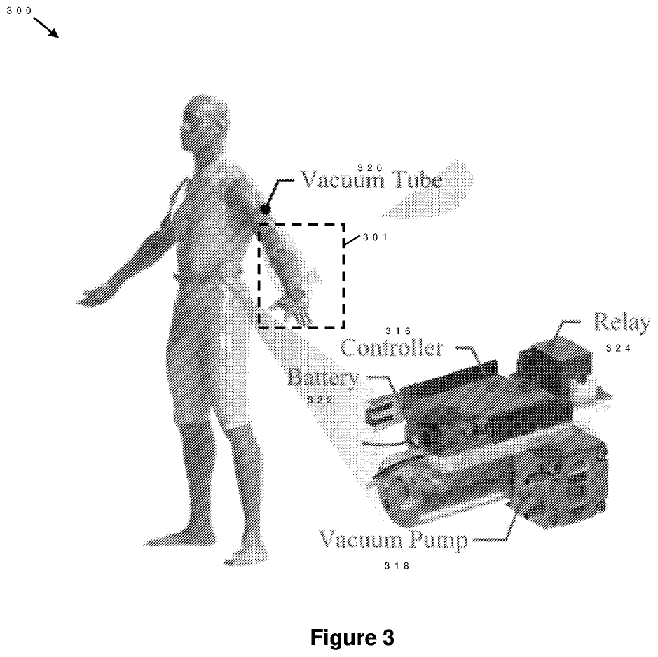

FIG. 3 depicts an exemplary embodiment 300 in which the exoskeleton 100 further comprises a vacuum pump 318 provided at the waist of the wearer and connected to other components 301 of the exoskeleton 100 provided at the arm and wrist. In this embodiment, the controller 316 may be provided together with the vacuum pump 318, along with auxiliary devices such as a battery 322 and a relay 324 mounted at the waist of the wearer. It is understandable to those skilled in the art that the vacuum pump 318 can be provided at other parts of the body of the wearer based on the practical needs.

In alternative embodiments, the vacuum pump may not form part of the exoskeleton 100, but is provided as an auxiliary device to the user to work in cooperation with the exoskeleton 100 to activate the variable stiffness unit 112 to provide the variable damping force to reduce the amplitude of the tremor.

Parameters of the embodiment 300 of the exoskeleton 100 are listed in Table 1.

TABLE 1
Parameters of the embodiment 300 of the exoskeleton 100

| Component | Parameters |
|---|---|
| Battery | Voltage: 12 V |
| IMU Sensor | Range of Euler angles: Roll: ±180°, Pitch: ±90°, Yaw: ±180° |
| | Sampling rate: Max 400 Hz |
| | Resolution: <0.01° |
| | Size: 39 mm × 39 mm × 8 mm |
| | Interface type: Bluetooth Classic 2.0 |
| Vacuum Pump | Option I¹*: Negative pressure: 88 kPa; Weight: 93 g |
| | Option II²*: Negative pressure: 53 kPa; Weight: 60 g |
| | Option III³*: Negative pressure: 40 kPa; Weight: 40 g |
| Variable stiffness unit | Weight: 277 g |
| Total weight | 455 g~515 g |

¹*The model number is PENGPU G2BL1288-2.
²*The model number is G1BK1253S.
³*The model number is OOH22H042.

A tremor may comprise muscle vibrations/motions in various directions. In some embodiments of the exoskeleton 100, the tremor is sensed in one or more of extension and flexion directions, abduction and adduction directions, and pronation and supination directions. In response, the variable damping force can be provided by the variable stiffness unit 112 in a direction along the direction of the sensed tremor.

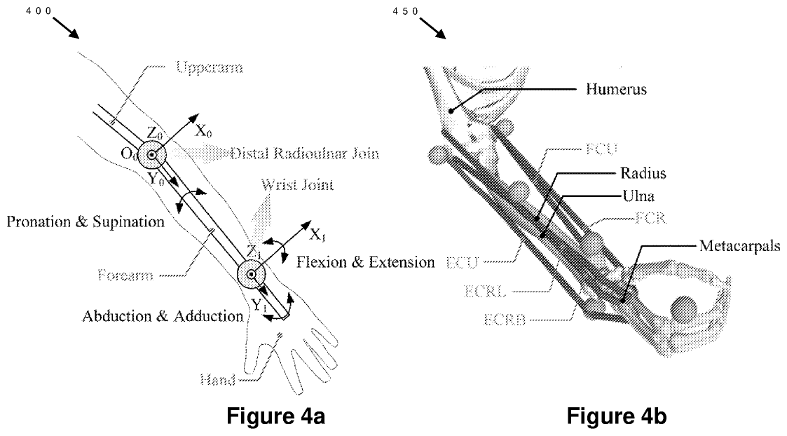

In order to effectively suppress tremors at wrist, the present specification has studied wrist tremor motion forms, i.e., the relationships between tremor amplitudes in each direction and forces or torques that cause the respective tremors. In this regard, FIG. 4a shows a diagram 400 of human wrist movements. FIG. 4b shows a diagram 450 illustrating major muscles and bones involved in wrist tremors.

In FIG. 4a, the diagram 400 depicts an anatomical skeletal structure of a human upper limb including a hand, a wrist joint, a forearm, a distal radioulnar joint, and an upper arm. The hand has two degrees of freedom (DoF) with respect to the forearm. The first DoF is rotation (flexion and extension) about the X₁ axis in the wrist joint coordinate system O₁-X₁Y₁Z₁. The typical range of rotatable angles about the X₁ axis is [−71°, 73°]. Rotation (abduction and adduction) about the Z axis is the second DoF with typical rotatable angles ranged between [−19°, 33°]. Rotation (pronation and supination) around the Y₀ axis in the distal radioulnar joint coordinate system O₀-X₀Y₀Z₀ has a third DoF relative to the upper arm, with a rotatable angular range of approximately [−86°, 71°]. A wrist tremor comprises combined motions in these three DoFs.

In FIG. 4b, the diagram 450 depicts Flexor Carpi Radialis (FCR), Flexor Carpi Ulnaris (FCU), Extensor Carpi Radialis Brevis (ECRB), Extensor Carpi Radialis Longus (ECRL), and Extensor Carpi Ulnaris (ECU), which are muscles involved in wrist tremors. All these five muscles work together to enable free movement of the wrist. The bones mainly include the humerus, radius, ulna, and metacarpal. Although the origins of wrist tremor in human bodies are still not clear, most studies show that it is related to human central nervous system concussion. In other words, the central nervous system electrically stimulates the relevant muscles to activate tremor.

According to Steven et. al, the wrist joint can be treated as a universal joint structure with two non-crossing axes. At the same time, the motion couplings between the wrist in flexion and extension as well as abduction and adduction are very small, which can be neglected. When the wrist joint is in its natural state, with the palm facing inward, the gravity factor will then reinforce the adduction direction. The set of kinetic equations incorporating the gravity factor can then be obtained, as shown in Equation (1).

$$\{ \begin{matrix}{T}_{α} - {T}_{P α} = {J}_{α} \ddot{α} \\ {T}_{β} + {T}_{g} - {T}_{P β} = {J}_{β} \ddot{β} \\ {T}_{γ} - {T}_{P γ} = {J}_{γ} \ddot{γ}\end{matrix} \qquad ( 1 )$$

where α, β, γ are wrist joint rotation angles in the flexion & extension, abduction & adduction, and pronation & supination directions, respectively. J_{α}, J_{β}, J_{γ} are moments of inertia of the wrist joint in the directions of flexion & extension, abduction & adduction, and pronation & supination directions, respectively. T_{α}, T_{β}, T_{γ} are active moments of the wrist joint in these three directions respectively, which can be considered as external torques caused by a force applied to the centre of the palm. Their expressions are shown in Equation (2). T_{g} is the gravitational moment acting on the wrist joint that causes the wrist to rotate in the adduction direction, and its calculation formula is shown in Equation (3).

$$\{ \begin{matrix}{T}_{Pi} = {K}_{Pi} \times i + {B}_{Pi} \times \frac{di}{dt} + {M}_{Pi} \\ {M}_{Pi} = {K}_{2 i} \frac{{e}^{\frac{{K}_{1 i}}{{K}_{3 i}} i}}{{e}^{{K}_{1 i}}} - {K}_{2 i} \frac{{e}^{- \frac{{K}_{1 i}}{{K}_{3 i}} i}}{{e}^{{K}_{1 i}}}\end{matrix} , i - α , β , γ \qquad ( 2 )$$

where K_{Pi}, B_{Pi}, K₁ᵢ, K₂ᵢ, and K₃ᵢ are joint fixation coefficients in the i direction.

$${T}_{g} = mgd cos β \qquad ( 3 )$$

where m is the mass of the palm, g is the gravitational constant, and dis the distance from the centre of mass of the palm to the wrist joint.

In the present specification, it is assumed that the active moment causing wrist tremor in flexion & extension, abduction & adduction directions is equivalent to external forces F_{α}, F_{β} applied at the centre of the palm. The active moment causing the forearm tremor in the direction of pronation & supination is equivalent to an external torque τ_{γ} applied at the centre of the palm. The relationships between F_{α}, F_{β}, τ_{γ} and the active torques T_{α}, T_{β}, T_{γ} then can be expressed as follows:

$$\{ \begin{matrix}{F}_{α} = \frac{{T}_{α}}{{l}_{α}} \\ {F}_{β} = \frac{{T}_{β}}{{l}_{β}} \\ {τ}_{γ} = {T}_{γ}\end{matrix} \qquad ( 4 )$$

Referring back to FIG. 1, when the exoskeleton 100 is worn on the wrist, the hand (not shown) only rotates to the forearm when the wrist produces tremors in the flexion & extension and abduction & adduction directions. The second support 104 can be fixed with the joint structure 106 at a forearm point C in this embodiment. The variable stiffness unit can completely cover the wrist. When the variable stiffness unit 112 is soft, the wrist point D can freely rotate against point C. When the variable stiffness unit 112 becomes rigid, the connection between points C and D can be considered as a stiff link which makes it difficult to rotate relative to the forearm anchor point C. As a result, tremors in the flexion & extension and abduction & adduction directions of the arm can be suppressed. The analysis in FIGS. 4a-4b shows that when a pronation & supination direction tremor occurs, the entire forearm rotates relative to the distal radioulnar joint. In this embodiment, the first support 102 can be fixed with the joint structure 106 at a point A of the upper arm, while the second support 104 is fixed with the joint structure 106 at the point C of the forearm near the elbow. The joint structure 106 can be a rigid joint that includes two arm members coupling with each other at point B. In this case, the rigid link BC has only one degree of freedom in comparison to the rigid link AB, which is the flexion-extension rotation to joint B. When the variable stiffness unit 112 is soft, the wrist point D can still rotate in the pronation & supination directions relative to point C. When the variable stiffness unit 112 is rigid, the BC and CD can be viewed as one rigid linkage. They can only be rotated in the flexion and extension directions relative to point B, not in the pronation and supination directions. As a result, forearm tremor in the pronation & supination directions can be limited.

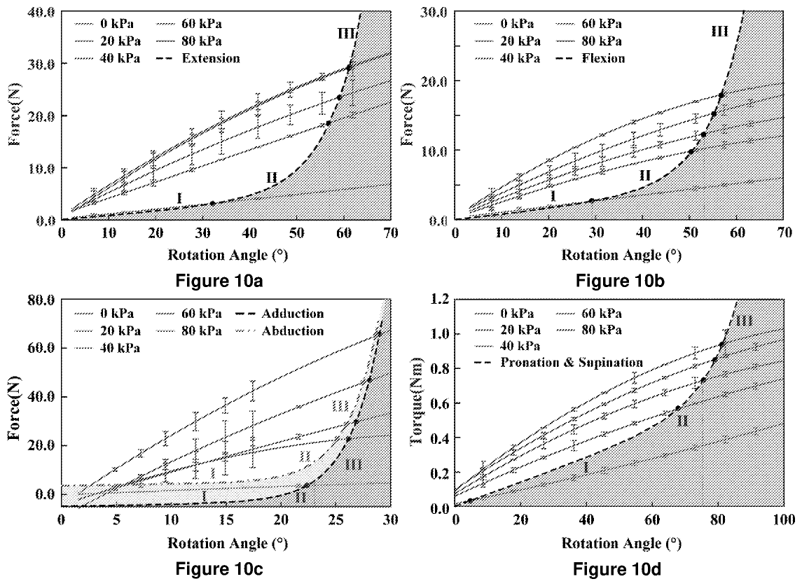

To quantify damping/resisting force ranges that the exoskeleton 100 needs to provide, and to balance between its lightweight and the maximum damping force, it is simulated in the present specification the relationships between equivalent forces in the flexion & extension and abduction & adduction directions, equivalent rotational torques triggering the rotation of pronation & supination, and the angle of rotation utilizing Equations (1) to (4). For the simulations in this specification, the Simulink module in the MATLAB 2021 Education Edition software is used. The relevant simulation parameters are shown in Table 2. The simulation results are shown in the dashed lines in FIGS. 10a to 10d to compare with experimental data.

TABLE 2
Simulation parameters

| Parameter | value | Parameter | value | Parameter | value | Parameter | value |
|---|---|---|---|---|---|---|---|
| J_{α,} | 1.30 g · m⁻² | B_{Pα} | 0.3 | K_{1β} | 6 | m | 0.445 kg |
| J_{β} | 0.87 g · m⁻² | B_{Pβ} | 0.3 | K_{2β} | 0.1 | g | 9.8 g · m⁻² |
| J_{γ} | 1.78 g · m⁻² | B_{Pγ} | 0.3 | K_{3β} | 0.3 | d | 0.086 m |
| K_{Pα} | 0.4 | K_{1α} | 6 | K_{1γ} | 6 | l_{α} | 0.09 m |
| K_{Pβ} | 0.3 | K_{2α} | 0.3 | K_{2γ} | 0.03 | l_{β} | 0.09 m |
| K_{Pγ} | 0.4 | K_{3α} | 0.8 | K_{3γ} | 1 | | |

It is desired for the exoskeleton 100 to be made of lightweight materials. In some embodiments, the variable stiffness unit 112 can be made of a chain mail-like structured fabric. The fabric comprises a plurality of interlocking particles that conform to the wearer of the exoskeleton 100 in an unjammed state when no pressure is applied to the fabric. The plurality of interlocking particles are configured to undergo a granular jamming transition to provide the variable damping force upon activation by the controller 116 in response to the tremor sensed by the sensor 114.

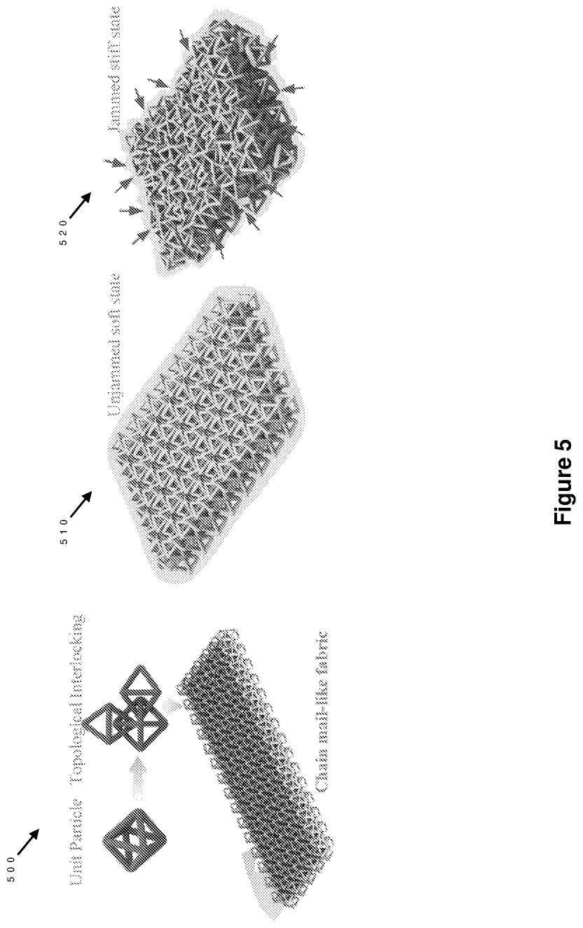

FIG. 5 shows a diagram 500 depicting an embodiment of such a chain mail-like structured fabric, a diagram 510 depicting a morphology of the chain mail-like structured fabric in an unjammed soft state, and a diagram 520 depicting a morphology of the chain mail-like structured fabric in a jammed stiff state.

In the diagram 500, a basic particle (or referred to as a unit particle) constructing the chain mail-like structured fabric is a hollow octahedron frame, which advantageously reduces overall density and weight while increasing contact between particles during jamming. The adjacent particles are interlocking and rotatable at 90° to each other, forming a topologically interlocked fabric structure.

In some embodiments, the unit particles and in turn, the chain mail-like structured fabric, can be 3D printed with a selective laser sintering (SLS) method from a high-strength lightweight nylon, making the exoskeleton 100 lightweight while meeting the strength requirements.

To increase the number of particle contacts during jamming, an embodiment of the present specification constructs a double layer of interlocking particles that are stacked on each other. The diagram 510 depicts the morphology of the chain mail-like structured fabric in the unjammed state without a negative pressure applied thereto. The double layer configuration allows the chain mail-like structured fabric to bend and fold easily, allowing the two layers to slide against each other while providing excellent conformability to the wrist. It is appreciable to those skilled in the art that the chain mail-like structured fabric is not limited to the double layer configuration as exemplified in FIG. 5. Modifications can be made to the number of layers, or the shape and structure of the unit particle based on the practical needs.

When a negative pressure is applied to the chain mail-like structured fabric, the interlocking particles undergo a granular jamming transition. In this scenario, the interlocking particles in the chain mail-like structured fabric form more contacts with their neighbours as shown in the diagram 520. When jammed, the bending stiffness of the chain mail-like structured fabric significantly increases within a fraction of a second. In this manner, the method 600 can further include a step in which the interlocking particles of the chain mail-like structured fabric are activated by the controller 116 to undergo a granular jamming transition to provide the variable damping force in response to the tremor sensed by the sensor 114.

FIG. 7 shows a schematic diagram of a control framework 700 to implement the exemplary method 600 of controlling the exoskeleton 100 as described herein. In this embodiment, the exoskeleton 100 comprises an inertial measurement unit (IMU) sensor 702 located on or proximate to a hand of a wearer of the exoskeleton 100, a data processing module 704, a controller 706, a relay 708, a vacuum pump 710, and a variable stiffness unit 712 that is located on or proximate to a wrist 714 of the wearer of the exoskeleton 100. It is understandable to those skilled in the art that, in alternative embodiments, the IMU sensor 702, the data processing module 704, the controller 706, the relay 708, and/or the vacuum pump 710 may not be part of the exoskeleton 100 but are provided as auxiliary devices working in conjunction with the exoskeleton 100.

In the control framework 700, the IMU sensor 702 senses and collects data on wrist tremors. The data is then filtered by the data processing module 704 using a second-order bandpass Butterworth filter with a passband of 2 Hz to 14 Hz. To determine the amplitude and/or frequency of a sensed tremor, a Fourier analysis is performed for the filtered data. The amplitude and/or frequency of the sensed tremor are then transmitted to the controller 706. Upon detecting such a tremor signal, the controller 706 controls the vacuum pump to start or stop by turning the relay 708 on or off based on the amplitude and/or frequency of the sensed tremor in the following manner.

When the controller 706 detects a tremor signal, the relay 708 is activated, and the vacuum pump 710 is turned on to activate the variable stiffness unit 712. Upon activation, the chain mail-like structured fabric in the variable stiffness unit 712 is jammed into a rigid state, thereby effectively suppressing the sensed tremor.

When the sensed tremor ceases, the controller 706 turns off the relay 708, the vacuum pump 710 is turned off, and the variable stiffness unit 712 gradually returns to its normal pressure state (1 atmosphere pressure). The chain mail-like structured fabric within the variable stiffness unit 712 gradually returns to its unjammed soft state. And the wrist 714 can regain its freedom of movement in all directions.

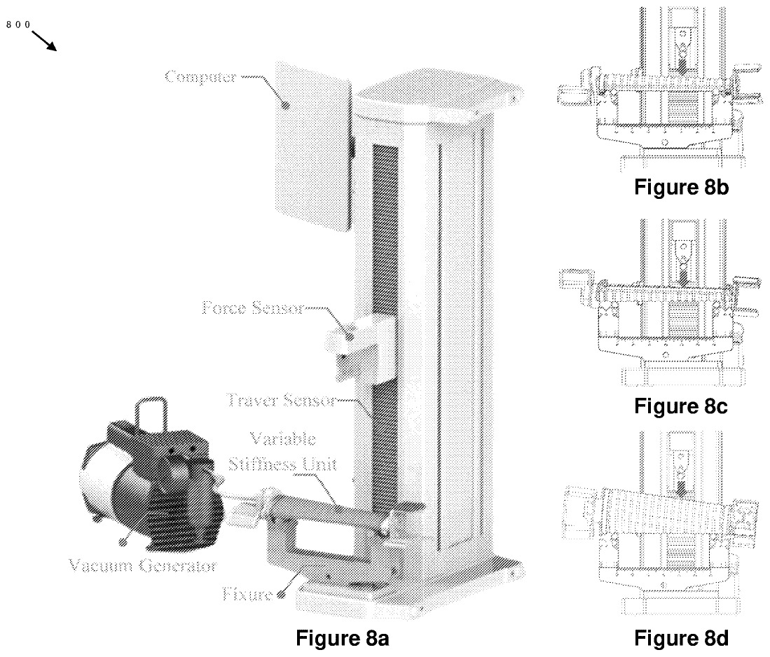

FIGS. 8a-8d, 9a-9b, 10a-10d, 11a-11f, 12 and 13a-13f depict experimental data that proves effectiveness of the damping force and/or torque provided by the exoskeleton 100 as described herein. The experimental data shows that the embodiments described herein produce an exoskeleton 100 that is both lightweight and effective to provide sufficient damping force and/or torque to suppress tremors in all three directions.

The experiments include bending and torsion tests that are conducted to obtain quantitative relationships between damping force/torque and deformation at different negative working pressures. Effective working regimes are calculated by comparing the damping force/torque to those obtained from the wrist dynamics model as set out in Equations (1) to (4). Details of the experimental data are as follows.

In further detail, FIG. 8a shows a schematic diagram of a Three-Point bending test apparatus 800 that can be used to test damping forces provided by various embodiments of the exoskeleton 100 in different directions.

FIGS. 8b, 8c and 8d respectively depict deformations of the variable stiffness unit 104 of various embodiments of the exoskeleton 100 during wrist extension, flexion, and abduction/adduction.

### Experimental Setup—Bending Test

In the bending test, the damping forces that the exoskeleton 100 can provide in different directions were tested using a 3-Point bending test equipment, as shown in Figure a(a). The variable stiffness unit 104 is attached to a fixture support at the bottom. A MARK-10 F305 mechanical tester with a force sensor (Range: 0 N-250 N, Resolution: 0.01 N) and a travel sensor (Range: 0 mm-475 mm, Resolution: 0.005 mm) is used for the test. In the test, a soft tube connects the variable stiffness unit 104 to a vacuum system. During the test, different pressure levels were applied to the exoskeleton 100 to trigger jamming transition. The exoskeleton 100 is then indented by the mechanical tester. The indenting position is chosen to be where the variable stiffness unit covers the wrist.

The exoskeleton 100 can bend in four directions: extension, flexion, abduction, and adduction. Since the structure of the variable stiffness unit is symmetrically distributed in abduction and adduction directions, only one bending direction needs to be measured in these two directions. Force and displacement relationships are tested in three different directions. FIGS. 8b, 8c and 8d depict the deformation of the variable stiffness unit during wrist extension, flexion, and abduction/adduction, respectively. Bending performance of the exoskeleton 100 is measured at negative pressures 0 kPa, 20 kPa, 40 kPa, 60 kPa, and 80 kPa. To obtain quasi-static bending results, the loading rate is set at 0.05 mm/s. The tests were repeated three times for each pressure, then averaged for results and standard deviations are calculated.

In the torsion test, a testing platform is designed and built to investigate the relationship between torsional torque and rotation angle.

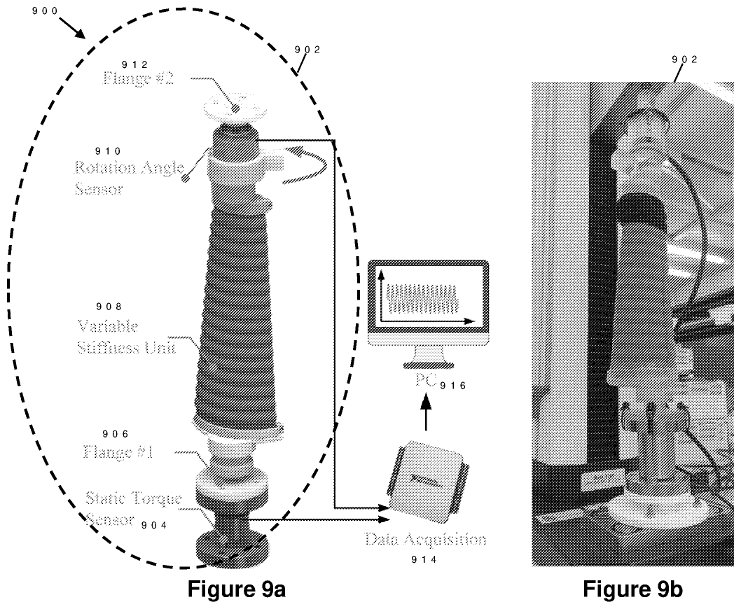

In further detail, FIG. 9a shows a schematic diagram of a torsional testing system 900 that can be used to test damping torque provided by the exoskeleton 100.

FIG. 9b shows an embodiment of a torsional testing platform 902 used in the torsional testing system 900.

### Experimental Setup—Torsion Test

The set-up schematic of the torsional testing system 900 is shown in FIG. 9a with an embodiment shown in FIG. 9b. It comprises a static torque sensor 904, flange #1 906, a variable stiffness unit 908, an angle sensor support, and flange #2 912. The bottom flange of the static torque sensor 904 is bolted to the table, and the top flange 912 is bolted to the fixing flange #1 906. The fixed flange #1 906 has a pitch hole and is interference fitted to bottom pitch shaft of the variable stiffness unit 908. Interference fit connects the top shaft of the variable stiffness unit 908 to the bottom hole of the angle sensor support. The angle sensor 910 is attached to the support, and its extension shaft is coupled to the top fixing structure via a coupling. The variable stiffness unit 908 is linked to a vacuum system via a soft tube.

The variable stiffness unit 908 was tested by applying different negative pressures to the structured fabric to trigger jamming transition and then twisting the structured fabric by rotating the angle sensor 910. During this process, the rotation angle and the output torque were recorded. The test was repeated three times for each pressure and averaged for the results, standard deviations were calculated based on the three tests.

### Results and Discussion of the Bending and Torsion Tests

FIGS. 10a-10d depict fitted curves of the bending and torsion test results as well as their errors. Shaded areas represent failure interval of the exoskeleton 100.

In further detail, FIG. 10a shows a graph diagram depicting bending and torsion test results of damping force against rotation angle in extension direction under different working negative pressures. FIG. 10b shows a graph diagram depicting bending and torsion test results of damping force against rotation angle in flexion direction under different working negative pressures. FIG. 10c shows a graph diagram depicting bending and torsion test results of damping force against rotation angle in abduction/adduction direction under different working negative pressures. FIG. 10d shows a graph diagram depicting bending and torsion test results of damping force against rotation angle in pronation/supination direction under different working negative pressures.

All the dashed lines in FIGS. 10a-10d are the results of rotation angle-force/torque simulation results utilizing mathematical models established in Equations (1)-(4).

Since the results shown in FIGS. 10a-10d have similar features, FIG. 10a is discussed in detail as a representative of the analysis, whereas FIGS. 10b-10d can be extended from FIG. 10a.

In FIG. 10a, the experimental results of damping force against rotation angle under different working negative pressures show that the damping force increases with increasing negative pressure, with a non-linear relationship. When the negative pressure increases from 0 kPa to 20 kPa, the damping force increases significantly. However, when the negative pressure increases from 20 kPa to 80 kPa, the damping force has a slower increase. In particular, when the negative pressure increases from 60 kPa to 80 kPa, there is almost no change in the damping force. This suggests that the exoskeleton 100's performance saturates at this pressure range.

Furthermore, in FIG. 10a, the simulation results of the damping force against rotation angle (the dashed line) are plotted. The simulation curve can be divided into three segments: The segment I has a very small slope, indicating that only a small force can cause a large wrist tremor amplitude in the extension direction. The transition segment is segment II, where the curve's slope gradually increases. When the curve reaches segment III, it becomes extremely sharp, which suggests slight wrist tremor amplitude increase requires a very large equivalent external force at this stage. The results in other directions shown in FIGS. 10b-10d show similar features and can be extended from this discussion.

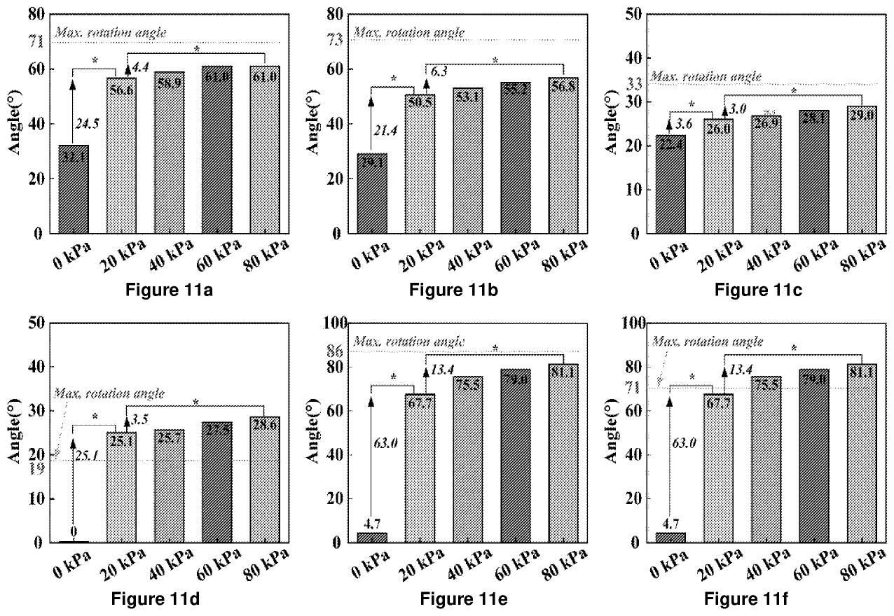

In order to quantitatively evaluate the tremor suppression capability of the exoskeleton 100, the indicator of critical tremor angle is introduced. It is defined as the intersection of the dashed line(s) in FIG. 10a-10d with the damping force test curves at various operating negative pressures. If the tremor amplitude is less than this angle (shown in the non-shaded portion of FIG. 10a-10d), the exoskeleton 100 can provide enough damping force to suppress the wrist tremor under this pressure. Otherwise, the damping force will be insufficient. Based on this, critical tremor angles at various negative pressures and directions are calculated, as shown in FIGS. 11a to 11f, in which the dashed line represents the greatest angle the wrist can reach in that direction.

In further detail, FIG. 11a shows a bar graph depicting critical tremor angles at various negative operating pressures in extension direction. FIG. 11b shows a bar graph depicting critical tremor angles at various negative operating pressures in flexion direction. FIG. 11c shows a bar graph depicting critical tremor angles at various negative operating pressures in abduction direction. FIG. 11d shows a bar graph depicting critical tremor angles at various negative operating pressures in adduction direction. FIG. 11e shows a bar graph depicting critical tremor angles at various negative operating pressures in pronation direction. FIG. 11f shows a bar graph depicting critical tremor angles at various negative operating pressures in supination direction.

According to FIGS. 11a-11f, it is shown that the maximum wrist rotation angle can be completely covered by the critical tremor angle produced by the exoskeleton 100 at 20 kPa and above in the adduction direction and at 40 kPa and above in the supination direction. When the working pressure reaches 80 kPa in the extension, flexion, abduction, and pronation directions, the critical tremor angle achieved by the exoskeleton 100 can reach as high as 85.9%, 77.80%, 81.8%, and 94.3% of the maximum angle of rotation respectively. In addition, the flexion direction has the lowest percentage of critical tremor angles, implying that it is less capable of limiting tremor in this direction, as expected by the user. Furthermore, when the working pressure increases from 0 kPa to 20 kPa in all directions except abduction, the critical tremor angle covered by the exoskeleton 100 increases significantly (by 21.4° to) 63.0°. However, when the working negative pressure was raised from 20 to 80 kPa, the critical tremor angle increase was only 3.0° to 13.5°. This also indicates that the exoskeleton 100's suppression performance saturates at certain pressure levels. Since achieving higher negative pressure requires significantly increasing the size and weight of the vacuum device, working at lower negative pressures will benefit system portability and miniaturization. As a result, choosing a working negative pressure limit of 20 kPa is more cost effective based on the experiments herein.

When determining the exoskeleton 100's working pressure, the user's actual needs will be considered. If the user has a low amplitude of tremor, working at 20 kPa negative pressure is advantageous for the small and light design of the exoskeleton 100. When selecting a commercial vacuum pump, the rated working pressure should be a little higher, ensuring that the exoskeleton 100 continuous working pressure can reach the set pressure. Based on the analysis of the commercial pump parameters shown in Table 2, the negative pressure limit of the variable stiffness device 112 is set at 20 kPa, and a vacuum pump with a working negative pressure of 40 kPa can provide a better balance between device performance and light weight. If the user has severe tremor, the working negative pressure can be set up to 80 kPa. Although this increases the overall weight of the exoskeleton 100, it can completely eliminate tremor in the direction of adduction and supination while greatly suppressing tremor in other directions.

### Prototype Performance Evaluation

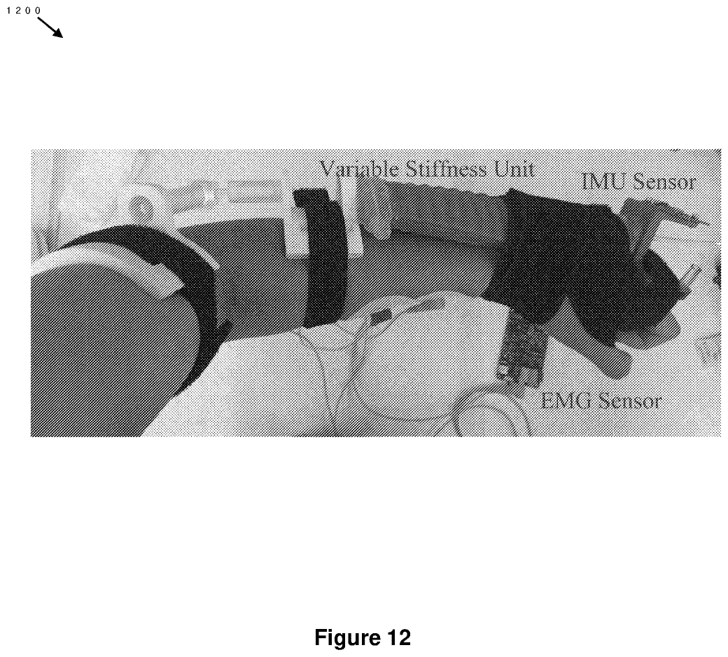

FIG. 12 shows a diagram 1200 depicting another embodiment of an exoskeleton in an experimental scene. Such an embodiment is a prototype exoskeleton.

The prototype was assembled after the damping force and torque experiments as described above. Two experiments were conducted to evaluate the prototype's performance on tremor suppression. In the first experiment, the prototype's ability to suppress hand tremors was tested in an active tremor case during which the wearer actively shakes the hand. During this experiment, the real-time response and signal acquisition capabilities of the prototype were also evaluated. In the second experiment, a tremor simulator device is constructed based on a motor driven linkage mechanism to test tremor suppression performance. This tremor simulator device is an important contribution of the present specification since it effectively generates tremors at controlled frequencies and amplitudes to demonstrate the effectiveness of the exoskeleton 100. For human experiments in this study, all subjects signed an informed consent form. The experiments performed in Nanyang Technological University were approved by the ethics committee. The experimental procedures adhered to the Helsinki Declaration.

### First Experiment-Active Tremor Experiment and Result Analysis

A young male participant was chosen to take part in the study for the active tremor experiment. The subject was 32 years old, with a hand length of approximately 18 cm. Wrist flexion & extension, abduction & adduction motions, and rotation relative to the distal radioulnar joint of forearm were all tested (pronation & supination). The participant was asked to swing his wrist or rotate his forearm at a frequency of 2 Hz to 6 Hz as much as possible during the experiment. The participant shakes for 10 seconds in the free state (without wearing exoskeleton 100). Motion data measured by IMU sensor is set as a reference group. The subject was then asked to wear the exoskeleton 100 while shaking at different negative pressures: 0 kPa, 40 kPa, and 80 kPa, respectively. Motion data measured by IMU sensor are set as an experimental group.

When the wrist trembled in the flexion & extension, abduction & adduction directions, the radial wrist flexors showed significant muscle activity, as seen in FIGS. 4a and 4b. When the forearm was rotated relative to the distal radioulnar joint, the triceps brachii muscle showed significant muscle activity. To minimize the participant's subjective influence, electromyographic (EMG) signals are collected from these two muscles in real time using EMG sensors. The sampling rate was set to 100 Hz. FIG. 12 depicts the prototype exoskeleton in the active tremor experiment.

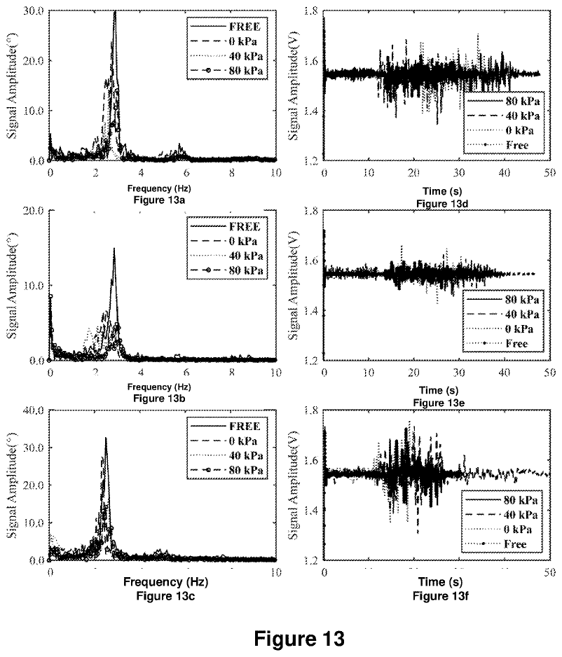

In the experiment, IMU sensors collected wrist tremor data at 0 kPa, 40 kPa, and 80 kPa in all directions, respectively. Furthermore, the collected data was Fourier transformed to obtain the dependence of the tremor amplitude on frequency, as shown in FIGS. 13a to 13c. FIGS. 13d to 13f show muscle surface EMG signals during wrist tremors under different negative working pressures.

FIGS. 13d to 13f show that the surface EMG signal amplitude was approximately 1.55V without tremor under different pressures. During tremor, the measured surface EMG signal values were 1.4V to 1.7V, 1.5V to 1.7V, and 1.3V to 1.8V in extension & flexion, abduction & adduction, and pronation & supination directions, with no significant difference in EMG signal amplitude. This result indicates that during the experiment, tremor force activation initiated by a participant was similar under different pressure conditions, which indicates that participants have less subjective influence.

Based on the analysis of FIGS. 13a to 13c, Table 3 shows the maximum tremor amplitudes of the wrist and forearm under different working negative pressures.

TABLE 3
Maximum amplitude of wrist tremor in three directions
under different negative pressures

| | Pressure | | | |
|---|---|---|---|---|
| | FREE | 0 kPa | 40 kPa | 80 kPa |
| Direction | Angle | | | |
| Extension & Flexion | 29.88° | 23.77° | 14.32° | 14.11° |
| Abduction & Adduction | 14.94° | 6.69° | 4.62° | 4.85° |
| Pronation & Supination | 32.64° | 27.70° | 16.61° | 14.00° |

To quantify the tremor suppression effect of this experiment, a tremor suppression efficiency η_{Exp1} is defined, as shown in Equation (5):

$${η}_{EXP 1} ( i ) = \frac{i {\vert}_{P}}{i {\vert}_{FREE}} \times 100 \% , i = α , β , γ \qquad ( 5 )$$

where i|_{P} denotes the experimentally determined tremor angle under pressure P. i|_{FREE} represents the experimental determination of the tremor angle under the free condition.

FIG. 13a shows a graph diagram depicting amplitude-frequency characteristics of wrist tremors in extension and flexion directions at various operating negative pressures. FIG. 13b shows a graph diagram depicting amplitude-frequency characteristics of wrist tremors in abduction and adduction directions at various operating negative pressures. FIG. 13c shows a graph diagram depicting amplitude-frequency characteristics of wrist tremors in pronation and supination directions at various operating negative pressures. FIG. 13d shows a graph diagram depicting electromyography (EMG) signals during extension and flexion tremor at various operating negative pressures. FIG. 13e shows a graph diagram depicting EMG signals during abduction and adduction tremor at various operating negative pressures. FIG. 13f shows a graph diagram depicting EMG signals during pronation and supination tremor at various operating negative pressures.

As shown in FIGS. 13a to 13f and Table 3, when the operating negative pressure of the exoskeleton 100 is set to 0 kPa, the tremor suppression efficiency η_{Exp1} in the extension & flexion, abduction & adduction, and pronation & supination directions are 20.45%, 55.22%, and 15.13%, respectively. When the working negative pressure is set to 40 kPa, η_{Exp1} in the above three directions are 52.07%, 69.07%, and 49.11%, respectively. The tremor suppression efficiency η_{Exp1} in the three directions increase to 52.78%, 67.54%, and 57.11% when the working negative pressure is set to 80 kPa. Based on these results, it shows that when the exoskeleton 100 is inactive (0 kPa), in the extension & flexion and pronation & supination directions, tremor suppression is minimal, and the exoskeleton 100 provides little resistance to wrist motion. When the exoskeleton 100 is activated at moderate pressure (40 kPa), the tremor can be suppressed significantly. When the exoskeleton 100 is fully activated (80 kPa), tremor suppression increases to a maximum, but the difference from 40 kPa is relatively small, indicating that further increasing negative pressure has a small effect on improving tremor suppression performance.

The above experiments indicate that the damping force and torque have a positive but non-linear relationship with the negative pressure. When the negative pressure exceeds 40 kPa, the increasing rate of the damping force and torque saturates significantly.

When the exoskeleton 100 is inactive (0 kPa), the vibration in abduction & adduction directions is reduced by roughly half compared to when it is not worn, indicating that the exoskeleton 100 still partly limits wrist motion even in the soft state. The results are consistent with the critical tremor angle experiments described with respect to FIGS. 10a-10d and FIGS. 11a-11f. It is noted that the daily movement of user is not significantly affected by the wrist movement in the abduction & adduction directions. When the exoskeleton 100 is fully activated (80 kPa), the tremor attenuation further increases, and the exoskeleton 100 appears to effectively suppress wrist tremors in the abduction & adduction directions.

### Second Experiment—Tremor Simulator and Experiment Results

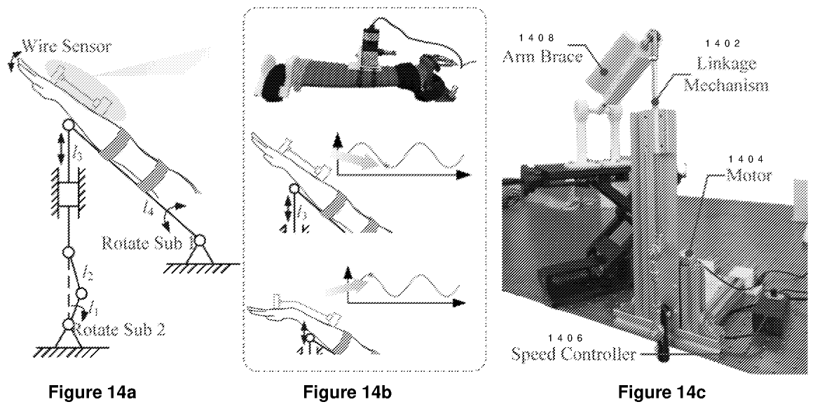

FIG. 14a shows a schematic diagram of a tremor simulator device, according to an embodiment. FIG. 14b shows an embodiment of a wire sensor and its testing principle. FIG. 14c shows an embodiment of the tremor simulator device.

In the embodiment shown in FIG. 14c, the tremor simulator device comprises a linkage mechanism 1402, a drive motor 1404, and a speed controller 1406. The linkage mechanism 1402 comprises a first rod and a second rod that are configured to rotate with respect to each other, and a third rod fixedly connected to the second rod. The third rod is configured to make a linear reciprocal motion with respect to the second rod. The speed controller 1406 is configured to control the drive motor to drive the first rod and the second rod to rotate and the third rod to make the linear reciprocal motion so as to cause a shaking at an end of a limb coupled to the tremor simulator device.

In some embodiments, the tremor simulator device further comprises a limb holder couplable with a limb. The linkage mechanism is rotatably connected to an end of the limb holder.

In some embodiments, the limb can be a forearm of a user. In this scenario, the limb holder is configured to be coupled to a forearm of a user. As exemplified in FIG. 14c, the limb holder is an arm brace 1408 in this scenario. It is understood that the limb holder can be coupled to other parts of the user based on the practical needs.

In some embodiments, the tremor simulator device further comprises a sensor configured to be placed on a hand of the user. The sensor is configured to measure a displacement of the hand in relation to the forearm of the user in extension and flexion directions under a predetermined value of the tremor at a predetermined frequency of the tremor. It is understood that the sensor can be placed on other parts of the user based on the practical needs.

For example, the predetermined frequency of the tremor can be in a low frequency range of 2.2 Hz to 2.7 Hz or 3.4 Hz to 3.7 Hz. It is understood that the predetermined frequency of the tremor can be in other frequency ranges.

During the test, the forearm of the user is restrained by the arm brace 1408. The speed controller controls the motor 1404 and drives the rods l₁ and l₂ to rotate, causing the rod l₃ to make a linear reciprocal motion. Simultaneously, when l₃ performs the linear reciprocal motion, it causes rod l₄ (the arm brace 1408) to rotate around Rotate Sub 1, causing the forearm to shake up and down. When the hand is completely relaxed, it will swing relative to the forearm due to inertia. As a result, hand tremors can be simulated.

In addition, a wire sensor is chosen to measure changes in displacement from the back of the hand to the forearm. During the test, one end of the wire sensor is attached to the forearm and the other to the back of the hand. FIG. 14b depicts the operating principle. If there is no wrist tremor, the back of the hand has a very small relative displacement with respect to the forearm, and the wire sensor has a small signal. If the wrist tremor is severe, the back of the hand will have a larger relative displacement change compared to the forearm, and the wire sensor will also output a higher signal.

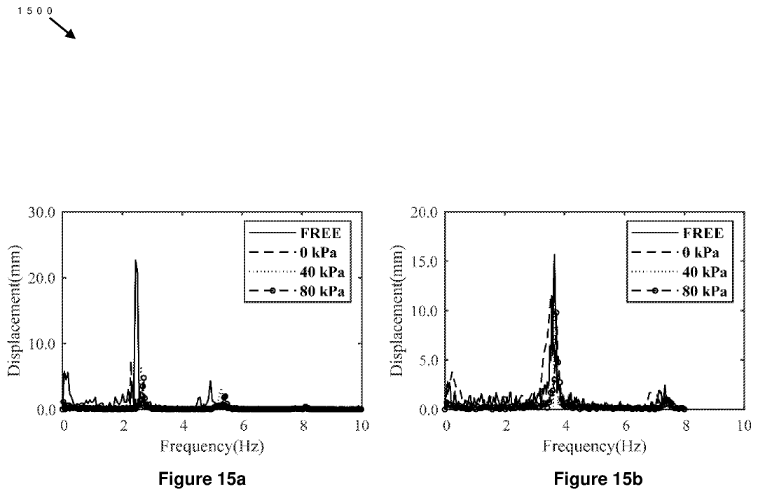

In the experiment, the relative displacement of the back of the hand in relation to the forearm in the extension and flexion directions is measured at 0 kPa, 40 kPa, and 80 kPa at lower (2.2 Hz to 2.7 Hz) and higher (3.4 Hz to 3.7 Hz) tremor frequencies, respectively. Furthermore, the collected data is Fourier transformed to obtain the variation of tremor amplitude with frequency, as shown in FIG. 15a and FIG. 15b.

FIG. 15a shows a graph diagram depicting maximum displacement variations of the wrist under different operating negative pressures for the wrist in the extension and flexion direction at a tremor frequency in the range of 2.2 Hz to 2.7 Hz.

FIG. 15b shows a graph diagram depicting maximum displacement variations of the wrist under different operating negative pressures for the wrist in the extension and flexion direction at a tremor frequency in the range of 3.4 Hz to 3.7 Hz.

By analysing FIGS. 15a and 15b, the maximum displacement variation of the wrist under different operating negative pressures can be obtained for the wrist in the extension & flexion direction at lower and higher tremor frequencies, as shown in Table 4.

TABLE 4
Maximum wrist tremor amplitude in extension & flexion direction
under different operating negative pressure and frequencies

| | Pressure | | | |
|---|---|---|---|---|
| | FREE | 0 kPa | 40 kPa | 80 kPa |
| Frequency | Displacement | | | |
| 2.2 Hz~2.7 Hz | 22.61 mm | 7.39 mm | 6.57 mm | 4.91 mm |
| 3.4 Hz~3.7 Hz | 19.56 mm | 10.53 mm | 6.15 mm | 5.71 mm |

In order to quantify the tremor suppression effect of this experiment, the tremor suppression efficiency η_{Exp2} is defined as shown in Equation (6):

$${η}_{EXP 2} ( L ) = \frac{L {\vert}_{P}}{L {\vert}_{FREE}} \times 100 \% \qquad ( 6 )$$

where L|_{P} represents the displacement measured by the displacement transducer under the P pressure. L|_{FREE} represents the displacement measured by the transducer under the free condition.

As shown in FIG. 15a and Table 4, when the tremor occurs at low frequency, the extension & flexion tremor suppression efficiency η_{Exp2} is approximately 67.31% when wearing the exoskeleton 100, and the negative pressure is 0 kPa. When the negative pressure is increased to 40 kPa, η_{Exp2} increases to 70.94%. When the negative pressure reaches 80 kPa, η_{Exp2} reaches 78.28%. The experimental results show that wearing the exoskeleton 100 under low-frequency tremor conditions significantly suppresses wrist tremors. As illustrated in FIG. 15b and Table 4, when wearing the exoskeleton 100 and experiencing a higher-frequency tremor, the extension & flexion η_{Exp2} is approximately 46.16%, and the negative pressure is 0 kPa. η_{Exp2} increases to approximately 68.56% when the negative pressure is increased to 40 kPa. When the negative pressure reaches 80 kPa, η_{Exp2} is approximately 70.81%. The experimental results show that wearing the exoskeleton 100 under low-frequency tremor conditions significantly suppresses wrist tremors and wearing the non-activated exoskeleton 100 has a small effect on wrist movement in the extension & flexion directions under high-frequency tremor conditions. When the exoskeleton 100 is fully activated, the wrist tremor can be effectively suppressed.

In view of the above experiments, it is noted that the exoskeleton 100 of the present application has a tremor suppression efficiency of about 52.78% to 78.28% in all directions. In terms of tremor suppression DoFs, the exoskeleton 100 of the present application can suppress tremors in all three directions. In terms of weight, the wrist part of the exoskeleton 100, i.e. the second support 104 that comprises the variable stiffness unit 112 is only about 277 g, which is relatively light and portable. Furthermore, the exoskeleton 100 of the present application can reach a much higher damping force compared to other existing prototypes. Thus, a compact, lightweight, conformable to the body, and comfortable to wear exoskeleton 100 is achieved by the present application.

TABLE 5
Performance parameters of the exoskeleton 100

| | Suppression | | | | | |
|---|---|---|---|---|---|---|
| Exoskeleton | Type | Weight (g) | DoF | Force | | Efficiency |
| VSW-Exo | Semi-Active | 443~582 | 3 ^{(WFE, WAA, WPS)} | ≤29.71 | N ^{(WF)}* | 52.78%— |
| (This Work) | | (Wrist: 277) | | ≤18.07 | N ^{(WE)} * | 78.28%^{(WFE)} |
| | | | | ≤7.3 | N ^{(WAD)} * | 67.54% ^{(WFE)} |
| | | | | ≤65.97 | N ^{(WAB)} * | 57.11% ^{(WFE)} |
| | | | | ≤0.93 | Nm ^{(WPS)} * | |

EFE: elbow flexion/extension,
FAA: forearm abduction/adduction,
FPS: forearm pronation/supination,
^{(WFE)} wrist flexion/extension,
^{(WF)} wrist flexion,
^{(WE)} wrist extension
^{(WPS)} wrist pronation/supination,
^{(WAA)} wrist abduction/adduction,
^{(WAB)} wrist adduction,
^{(WAD)} wrist abduction.
* The damping force is obtained based on the analysis in FIGS. 10a-10d

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

What is claimed is:

1. An exoskeleton, comprising:
a first support;
a second support;
a joint structure rotatably connecting the first support and the second support; and
a controller,
wherein the second support comprises:
a first anchor component connecting to the joint structure,
a second anchor component, and
a sensor coupled to the controller,
wherein the first anchor component and the second anchor component are connected by a variable stiffness unit coupled to the controller, and
wherein the variable stiffness unit is configured to provide a variable damping force determined by the controller to reduce an amplitude of a tremor sensed by the sensor.

2. The exoskeleton according to claim 1, further comprising:
a vacuum pump, wherein, in response to detection of a tremor signal received by the controller from the sensor upon sensing the tremor, the controller is configured to control the vacuum pump to activate the variable stiffness unit to provide the variable damping force.

3. The exoskeleton according to claim 1,
wherein the first support is couplable to an upper arm of a wearer of the exoskeleton,
wherein the first anchor component of the second support is couplable to a forearm of the wearer of the exoskeleton,
wherein the second anchor component of the second support is couplable to a hand of the wearer of the exoskeleton and the sensor is configured to be located on or proximate to the hand, and
wherein the variable stiffness unit is located at or proximate to a wrist of the wearer of the exoskeleton, spanning from the first anchor component to the second anchor component.

4. The exoskeleton according to claim 1,
wherein the tremor is sensed in one or more of extension and flexion directions, abduction and adduction directions, and pronation and supination directions, and
wherein the variable damping force is provided in a direction along the direction of the sensed tremor.

5. The exoskeleton according to claim 2,
wherein the variable stiffness unit is made of a chain mail-like structured fabric,
wherein the fabric comprises a plurality of interlocking particles that conform to the wearer of the exoskeleton in an unjammed state when no pressure is applied to the fabric, and
wherein the plurality of interlocking particles are configured to undergo a granular jamming transition to provide the variable damping force upon activation by the controller in response to the tremor sensed by the sensor.

6. The exoskeleton according to claim 5,
wherein the chain mail-like structured fabric is 3D printed with a selective laser sintering (SLS) method from a high-strength lightweight nylon.

7. A method of controlling an exoskeleton, the exoskeleton comprising a first support, a second support, a joint structure rotatably connecting the first support and the second support, and a controller, wherein the second support comprises a first anchor component connecting to the joint structure, a second anchor component, and a sensor coupled to the controller, and
wherein the first anchor component and the second anchor component are connected by a variable stiffness unit coupled to the controller,
wherein the method comprises:
sensing a tremor by the sensor,
determining a variable damping force by the controller in response to the sensed tremor, and
providing the variable damping force by the variable stiffness unit to reduce an amplitude of the tremor.

8. The method according to claim 7, wherein the exoskeleton further comprises a vacuum pump, and wherein the method further comprises:
detecting, by the controller, a tremor signal received from the sensor upon sensing of the tremor; and
controlling, by the controller, the vacuum pump to activate the variable stiffness unit to provide the variable damping force.

9. The method according to claim 7, further comprising:
coupling the first support at an upper arm of a wearer of the exoskeleton;
coupling the first anchor component of the second support at a forearm of the wearer of the exoskeleton;
coupling the second anchor component of the second support at a hand of the wearer of the exoskeleton such that the sensor is located at or proximate to the hand; and
locating the variable stiffness unit at or proximate to a wrist of the wearer of the exoskeleton, spanning from the first anchor component to the second anchor component.

10. The method according to claim 7, further comprising:
in response to the sensor sensing the tremor in one or more of extension and flexion directions, abduction and adduction directions, and pronation and supination directions, providing, by the variable stiffness unit, the variable damping force in a direction along the direction where the tremor is sensed.

11. The method according to claim 8,
wherein the variable stiffness unit is made of a chain mail-like structured fabric, and wherein the fabric comprises a plurality of interlocking particles that conform to the wearer of the exoskeleton in an unjammed state when no pressure is applied to the fabric, and wherein the method further comprises:
activating, by the controller, the plurality of interlocking particles to undergo a granular jamming transition to provide the variable damping force in response to the tremor sensed by the sensor.

12. A tremor simulator device, comprising:
a linkage mechanism;
a drive motor; and
a speed controller,
wherein the linkage mechanism comprises a first rod and second rod that are configured to rotate with respect to each other, and a third rod fixedly connected to the second rod, wherein the third rod is configured to make a linear reciprocal motion with respect to the second rod, and
wherein the speed controller is configured to control the drive motor to drive the first rod and the second rod to rotate and the third rod to make the linear reciprocal motion so as to cause a shaking at an end of a limb coupled to the tremor simulator device.

13. The tremor simulator device according to claim 12, further comprising:
a limb holder couplable with a limb, wherein the linkage mechanism is rotatably connected to an end of the limb holder.

14. The tremor simulator device according to claim 13, wherein the limb holder is configured to be coupled to a forearm of a user

15. The tremor simulator device according to claim 14, further comprising:
a sensor configured to be placed on a hand of the user, wherein the sensor is configured to measure a displacement of the hand in relation to the forearm of the user in extension and flexion directions under a predetermined value of the tremor at a predetermined frequency of the tremor.

16. The tremor simulator device according to claim 15, wherein the predetermined frequency of the tremor is in a low frequency range of 2.2 Hz to 2.7 Hz.

17. The tremor simulator device according to claim 15, wherein the predetermined frequency of the tremor is in a low frequency range of 3.4 Hz to 3.7 Hz.
